(19) United States
(12) Patent Application Publication
(10) Pub. No.: US 2025/0076297 A1
(43) Pub. Date: Mar. 6, 2025

(54) COMPOSITIONS AND METHODS FOR PLIMB-MEDIATED RADICAL LABELING IN VIVO

(71) Applicant: Immuto Scientific, Madison, WI (US)

(72) Inventors: Daniel Benjamin, Madison, WI (US); Faraz Choudhury, Fitchburg, WI (US); Jonathan Schmitz, Madison, WI (US); James Dowell, Middleton, WI (US); Alexis Lawton, Madison, WI (US); Samantha Knott, Madison, WI (US)

(21) Appl. No.: 18/817,445

(22) Filed: Aug. 28, 2024

Related U.S. Application Data
(60) Provisional application No. 63/536,873, filed on Sep. 6, 2023.

(51) Int. Cl. G01N 33/569; G01N 33/542; G01N 33/68

(52) U.S. Cl. CPC G01N 33/56966; G01N 33/542; G01N 33/6848; G01N 2458/15

(57) ABSTRACT

Provided herein are materials and methods for in vivo labeling of membrane proteins using plasma-induced modification of biomolecules (PLIMB).

### PRIORITY STATEMENT

This application claims priority to U.S. Provisional Patent Application No. 63/536,873, filed Sep. 6, 2023, the entire contents of which are incorporated herein by reference for all purposes.

### TECHNICAL FIELD

Provided herein are materials and methods for plasma-induced in vivo labeling of cell membrane proteins with one or more radicals.

### BACKGROUND

Chemical labeling of proteins coupled to mass spectrometry (MS), such as hydrogen-deuterium exchange (HDX) and radical footprinting, are a family of techniques which have been fundamental for the understanding of protein higher order structure (HOS) and the field of structural biology. The large sample requirements, protein size limitations and time for crystallization of high-resolution techniques, such as cryogenic electron microscopy (cryo-EM) and x-ray crystallography, often make highly dynamic protein samples less amenable to study. Although footprinting cannot provide the atomic resolution of crystallography, it is considerably higher throughput. Additionally, the resolution from a footprinting experiment approaches single amino acid level and rivals throughput of techniques such as peptide scanning or epitope binning, which are often limited to the peptide level. However, adopting these techniques for in vivo radical labeling presents unique analytical challenges. Membrane protein HOS and binding information remains an unmet challenge, and HRF has not been successfully used to determine the epitope region of binding partners in vivo. Accordingly, there remains a need for effective labeling of biological molecules in vivo.

### SUMMARY

Provided herein are methods for radical production and labeling of proteins in live cells.

In some aspects, provided herein is a method for in vivo labeling of cell membrane proteins with hydroxyl radicals, the method comprising: providing a sample comprising one or more live cells; and generating a plasma in the sample, thereby producing hydroxyl radicals in the sample which interact with cell membrane proteins associated with the one or more live cells, thereby labeling the cell membrane proteins in vivo.

In some embodiments, the sample comprises a phosphate buffer. In some embodiments, the buffer comprises phosphate buffered saline (PBS) or sodium phosphate buffer. In some embodiments, the sample comprises 1 mM to 500 mM PBS or sodium phosphate buffer. In some embodiments, the sample comprises 1 mM to 50 mM PBS or sodium phosphate buffer. In some embodiments, the sample comprises 5 mM PBS or sodium phosphate buffer. In some embodiments, the sample comprises a HEPES buffer.

In some embodiments, the sample additionally comprises a salt. For example, in some embodiments the salt comprises sodium chloride (NaCl), calcium chloride (CaCl₂)), potassium chloride (KCl), or sodium bicarbonate (NaHCO₃). In some embodiments, comprises salt at a concentration such that the sample is isotonic with the live cells. In some embodiments, the sample comprises the salt at a concentration of 100 μM to 1M. In some embodiments, sample comprises the salt at a concentration of 5 mM to 250 mM. In some embodiments, sample comprises the salt at a concentration of 25 mM to 150 mM.

In some embodiments, the plasma is generated for 1 ns to 72 hours. In some embodiments, the plasma is generated for 5 seconds to 120 seconds. In some embodiments, the plasma is generated for 60 seconds. In some embodiments, the plasma is generated from a plasma source point of a plasma electrode, wherein the plasma source point is submerged within the sample. In some embodiments, the plasma source point is submerged within the sample at a depth of 5-25 mm. In some embodiments, the plasma source point is submerged within the sample at a depth of 15 mm.

In some embodiments, the method further comprises quenching the sample with a solution comprising a radical quencher following generating the plasma in the sample.

In some embodiments, the sample further comprises a radical precursor for a second radical other than hydroxyl radicals, and wherein generating a plasma in the sample converts the radical precursor into the second radical which additionally interacts with and labels the cell membrane proteins in vivo.

In some embodiments, the method further comprises identifying labeling of the biological molecule with the hydroxyl radicals using mass spectrometry.

### BRIEF DESCRIPTION OF THE DRAWINGS

FIG. 1A is a bar graph showing plasma induced labeling of biomolecules (PLIMB) dependent oxidation of methionine 122 from CTLA4-FLAG immunoprecipitated from FreeStyle™ 293-F cells. The peak intensity from LC-MS/MS analysis of OH modification of methionine 122 from CTLA4-FLAG immunoprecipitated from FreeStyle™ 293-F cells was calculated for three independent biological in PLIMB treated cells (60 seconds) and a non-PLIMB control. Two-tailed two-sample equal variance (homoscedastic) Student's t-tests were performed to detect statistically significant differences in percent intensity. Asterisks indicate a statistically significant change in percent modification across conditions (p<0.001).

FIG. 1B is a bar graph showing plasma induced labeling of biomolecules (PLIMB) dependent oxidation of cysteine 58 from CTLA4-FLAG immunoprecipitated from FreeStyle™ 293-F cells. The peak intensity from LC-MS/MS analysis of OH modification of cysteine 58 from CTLA4-FLAG immunoprecipitated from FreeStyle™ 293-F cells was calculated for three independent biological in PLIMB treated cells (60 seconds) and a non-PLIMB control. Two-tailed two-sample equal variance (homoscedastic) Student's t-tests were performed to detect statistically significant differences in percent intensity. Asterisks indicate a statistically significant change in percent modification across conditions (p<0.01).

### DETAILED DESCRIPTION

Section headings as used in this section and the entire disclosure herein are merely for organizational purposes and are not intended to be limiting.

All publications, patent applications, patents and other references mentioned herein are incorporated by reference in their entirety.

### 1. Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

### 2. Methods for Labeling Membrane Proteins In Vivo

Radical based “protein footprinting” provides a healthy balance between throughput and resolution. Protein footprinting approaches probe changes in structure by mapping solvent-accessible surface area (SASA) via labeling of amino acid sidechains in solution with a chemical moiety, followed by quantification using MS. Applications of protein footprinting include antibody epitope localization, where regions of protein interactions are characterized by changes in SASA between two different conditions (e.g., bound and unbound to the antigen's corresponding antibody). A region showing protection, or a decreased level of labeling in the bound condition versus unbound, can therefore be an indication a binding region. However, to date radical footprinting has not been employed successfully for in vivo labeling of cell membrane proteins, which account for a large portion of the proteome and are the targets for many drugs in development. Mapping the HOS of membrane proteins using protein footprinting has presented a significant challenge due to the inherent insolubility and multiple transmembrane domains. This disclosure addresses this need and provides a platform for effective labeling of cell membrane proteins in vivo.

In some aspects, provided herein is a method for in vivo labeling of cell membrane proteins. The terms “membrane protein” or “cell membrane protein” are used interchangeably herein and refer to a protein associated with the membrane of a cell. In some embodiments, the protein is attached to the extracellular surface of the cell membrane. In some embodiments, the protein has one or more transmembrane domains. In some embodiments, provided herein is a method for in vivo labeling of cell membrane proteins with one or more types of radicals involving providing a sample comprising live cells and generating a plasma in the sample, thereby generating one or more radical types from radical precursor(s) present in the sample which interact with and label membrane proteins associated with the live cells.

In some embodiments, provided herein is a method for in vivo labeling of cell membrane proteins with hydroxyl radicals, the method comprising providing a sample comprising one or more live cells, and generating a plasma in the sample, thereby producing hydroxyl radicals in the sample. The hydroxyl radicals produced in the sample interact with cell membrane proteins associated with the one or more live cells, thereby labeling the cell membrane proteins in vivo. The methods provided herein thus are used to label cell membrane proteins in their native environment: live, intact cells. Accordingly, the methods provided herein are used successfully to label cell membrane proteins and investigate HOS in the native environment, a need which was previously unmet by existing protein-labeling technologies.

In some embodiments, the sample comprises live cells. In some embodiments, the live cells are substantially intact. In other words, in some embodiments the cell membranes of the live cells are substantially intact and have not been ruptured or permeabilized prior to generating a plasma in the sample.

In some embodiments, the sample comprises live cells and a buffer. The buffer can comprise any suitable buffer to maintain cell viability while providing the conditions suitable for generation of radicals. Exemplary buffers include, for example, phosphate buffered saline solution, tris(hydroxymethyl)aminomethane (tris), tris hydrochloric acid, ammonium bicarbonate, 4-(2-hydroxyethyl)-1-piperazineethanesulfonic acid (HEPES), 3-(N-morpholino)propanesulfonic acid (MOPS), 2-(N-morpholino)ethanesulfonic acid (MES), 2,2-Bis(hydroxymethyl)-2,2′,2″-nitrilotriethanol (bis-tris), N-(2-Acetamido)iminodiacetic acid (ADA), piperazine-N,N′-bis(2-ethanesulfonic acid) (PIPES). N-(2-Acetamido)-2-aminoethanesulfonic acid (ACES), 3-(N-morpholinyl)-2-hydroxypropanesulfonic acid sodium salt (MOPSO), 1,3-bis(tris(hydroxymethyl)methylamino)propane (bis-tris propane), N,N-Bis(2-hydroxyethyl)-2-aminoethanesulfonic acid (BES), 2-[[1,3-dihydroxy-2-(hydroxymethyl)propan-2-yl]amino]ethanesulfonic acid (TES), 3-(Bis(2-hydroxyethyl)amino)-2-hydroxypropane-1-sulfonic acid (DIPSO), 3-[[1,3-dihydroxy-2-(hydroxymethyl)propan-2-yl]amino]-2-hydroxypropane-1-sulfonic acid (TAPSO), Trizma, piperazine-1,4-bis(2-hydroxypropanesulfonic acid) dihydrate (POPSO), 3-[4-(2-Hydroxyethyl)-1-piperazinyl]propanesulfonic acid (HEPPS), N-(2-Hydroxy-1,1-bis(hydroxymethyl) ethyl) glycine (TRICINE) glycylglycine (GLY-GLY), 2-(Bis(2-hydroxyethyl) amino) acetic acid (BICINE), N-(2-hydroxyethyl)piperazine-N′-(4-butanesulfonic acid) (HEPBS), 3-[[1,3-dihydroxy-2-(hydroxymethyl)propan-2-yl]amino]propane-1-sulfonic acid (TAPS), 2-amino-2-methyl-1,3-propanediol (AMPD), N-(1,1-Dimethyl-2-hydroxyethyl)-3-amino-2-hydroxypropanesulfonic acid (AMPSO), N-cyclohexyl-2-aminoethanesulfonic acid (CHES), N-cyclohexyl-2-hydroxyl-3-aminopropanesulfonic acid (CAPSO), 1-amino-2-methyl-1-propanol (AMP), N-cyclohexyl-3-aminopropanesulfonic acid (CAPS), 4-(cyclohexylamino)-1-butanesulfonic acid (CABS), Lysogeny broth (LB) or other nutrient growth media, anything defined as a ‘biological buffer’, a biologically or physiologically-relevant salt, combinations thereof, and the like. In some embodiments, the buffer has a pH value of between 1 and 14, including but not limited to, a pH of between 3 and 9, or a pH of between 4 and 8.

In some embodiments, the sample comprises a phosphate buffer. For example, in some embodiments the buffer comprises phosphate buffered saline (PBS) or sodium phosphate buffer. In some embodiments, the sample comprises 1 mM to 500 mM PBS or sodium phosphate buffer. For example, in some embodiments the sample comprises 1 mM to 500 mM, 1.5 mM to 400 mM, 2 mM to 300 mM, 2.5 mM to 200 mM, 3 mM to about 100 mM, 3.5 mM to about 50 mM, 4 mM to about 25 mM, or about 5 mM to about 15 mM PBS or sodium phosphate buffer. In some embodiments, the sample comprises 1 mM-50 mM PBS or sodium phosphate buffer. For example, in some embodiments the sample comprises 1 mM to 50 mM, 2 mM to 40 mM, 3 mM to 30 mM, 4 mM to 25 mM, or 5 mM to 20 mM PBS or sodium phosphate buffer. In some embodiments, the sample comprises 5 mM PBS or sodium phosphate buffer. In some embodiments, the PBS or phosphate buffer has a pH of 7.4

In some embodiments, the sample comprises HEPES buffer. In some embodiments, the sample comprises 5-50 mM HEPES buffer. For example, in some embodiments the sample comprises 5 mM HEPES, 10 mM HEPES, 15 mM HEPES, 20 mM HEPES, 25 mM HEPES, 30 mM HEPES, 35 mM HEPES, 40 mM HEPES, 45 mM HEPES, or 50 mM HEPES. In some embodiments, the HEPES buffer has a pH of 6.0. In some embodiments, the HEPES buffer has a pH of 7.4.

In some embodiments, the method of labeling cell membrane proteins in vivo comprises generating a plasma in a sample comprising live cells. Exemplary systems and methods for generating plasma are described in PCT Patent Publication No. WO2017201457A1, the entire content of which is incorporated herein by reference for all purposes. In some embodiments, plasma is generated by a plasma electrode, also referred to as a plasma needle. In some embodiments, the plasma is generated by a plasma electrode comprising a plasma source point. The “plasma source point” refers to the point on the electrode from which the plasma emerges. In some embodiments, the plasma electrode comprises a dielectric coating that prevents direct contact between the plasma electrode and the sample. In some embodiments, the dielectric coating covers at least the plasma source point. In some embodiments, the plasma electrode is a component of a plasma jet, which refers to a device which generates a plasma within a first space and propels the generated plasma towards a target (e.g. the sample) by movement of gas through the jet and the shaping of the plasma jet. In some embodiments, the gas is argon. In some embodiments, the gas is helium. Exemplary plasma jets are described in Kim. Y. H., et al. Plasma Chem Plasma Process 34, 457-472 (2014). J Voráč et al. Plasma Sources Sci. Technol. 22 (2013), Ghimire et al., AIP Advances 8, 075021 (2018), Tampieri et al., Anal. Chem. 2021, 93, 8, 3666-3670, and Chauvin, J. et al. Sci Rep 7, 4562 (2017), each of which is incorporated herein by reference in their entireties.

In some embodiments, plasma is generated by a system including a ground electrode, a plasma electrode, and a power supply. In some embodiments, electrical power from the power supply is used to generate a high voltage signal, whether alone or in combination with an amplifier. In some embodiments, plasma is formed when the voltage between the plasma electrode and the ground electrode exceeds a given value. The value may be dependent on the voltage magnitude, the frequency of the signal, and/or the space between the plasma electrode and the ground electrode.

In some embodiments, plasma can be generated by a voltage of between 1 volt (V) and 1 megavolt (MV). For example, in some embodiments plasma is generated by a voltage between 1V and 1 MV, between 100V and 750 kilovolts (kV), between 500 V and 500 kV, between 1 kV and 100 kV, between 1 kV and 50 kV, or between 5 kV and 15 kV.

In some embodiments, the plasma source point of the plasma electrode is placed within the sample (e.g. is submerged). In some embodiments, the plasma source point is submerged within the sample to a depth of 5-25 mm. In some embodiments, the plasma source point is submerged within the sample to a depth of 10-20 mm. In some embodiments, the plasma source point is submerged within the sample to a depth of 15 mm.

In some embodiments, the plasma source point of the plasma electrode is placed at the interface of a gas and the sample. In some embodiments, plasma is generated from a plasma source point placed at an interface of a gas and the sample, and the plasma then travels from the gas to the sample. In some embodiments, the gas is air. In some embodiments, the gas (e.g. air) refers to gas comprising both oxygen and nitrogen. In some embodiments, the sample is in contact with a fluid, and plasma is generated at the interface of fluid and the sample. In some embodiments, the sample is in contact with a fluid, and plasma is generated at the interface of gas (e.g. air) and the fluid, which plasma then passes through the fluid and into the sample. In some embodiments, the plasma source point is placed (and thus plasma is generated) within 10 cm of the sample. In some embodiments, the plasma source point is placed within 3 cm of the sample. The plasma will be generated in the gas (e.g. air) and pass onto the sample. For example, in some embodiments the plasma electrode is placed at the interface of gas and the sample, within 3 cm, within 2.5 cm, within 2 cm, within 1.5 cm, within 1 cm, or within 0.5 cm of the sample. “Generating a plasma in a sample” is inclusive of any of the above-described mechanisms.

The plasma can be generated for any suitable duration of time. In some embodiments, the plasma is generated for generated for 1 ns to 72 hours. For example, in some embodiments the plasma is generated for 1 ns to 72 hours, 100 ns to 24 hours, 1500 ns to 12 hours, 1 second to 6 hours, 1.5 seconds to 1 hour, 2 seconds to 30 minutes, 3 seconds to 10 minutes, 4 seconds to 5 minutes, or 5 seconds to 2 minutes. In some embodiments, the plasma is generated for 5 seconds to 120 seconds. For example, in some embodiments the plasma is generated for 5 seconds to 120 seconds, 10 seconds to 100 seconds, 20 seconds to 90 seconds, 30 seconds to 80 seconds, 25 seconds to 75 seconds, 30 seconds to 70 seconds, 40 seconds to 60 seconds, or about 50 seconds. In some embodiments, the plasma is generated for 60 seconds. The duration of plasma generation can be controlled by the power supply, which can turn off power to the plasma electrode after the desired duration of plasma generation has occurred.

In some embodiments, the sample comprises PBS or a phosphate buffer, which provides a suitable environment to maintain cell viability and provides water as a radical precursor for generation of hydroxyl radicals from plasma. In some embodiments, the sample additionally comprises one or more additional radical precursors for generation of additional radical types (e.g. other than hydroxyl radicals). For example, in some embodiments the sample additionally comprises a radical precursor for a second radical other than hydroxyl radicals, and wherein generating a plasma in the sample converts the radical precursor into the second radical which additionally interacts with and labels the cell membrane proteins in vivo. In some embodiments, the second radical is a hydrogen radical (·H), a nitrite or nitrogen dioxide radical (·NO₂), a nitrate radical (·NO₃), a peroxide radical (·OOH), a trifluoromethyl radical (·CF3), or combinations thereof. In some embodiments, the sample comprises precursors for a hydrogen radical (·H), a nitrite or nitrogen dioxide radical (·NO₂), a nitrate radical (·NO₃), a peroxide radical (·OOH), a trifluoromethyl radical (·CF3), or combinations thereof. Examples of the radical precursors include, but are not limited to, water, hydrogen peroxide, hydrogen gas, nitrite, nitrogen dioxide, nitrate, sodium triflinate, or combinations thereof. Exemplary gases that serve as precursors for trifluoromethyl radicals include hexafluoroethane (C₂F₆), trifluoromethane (aka fluoroform, HCF₃), trifluoronitrosomethane (CF₃NO), and hexafluoroazomethane (CF₃CN₂CF₃). In some embodiments, the second radical type is CF3, and the sample comprises the radical precursor sodium triflinate. In some embodiments, the second radical type is CF3, and the sample comprises sodium triflinate and a peroxide. In some embodiments, the second radical type is CF3, and the sample comprises sodium triflinate and tert-butyl hydroperoxide (TBHP). In some embodiments, the second radical type is CF3, and the sample comprises sodium triflinate and hydrogen peroxide.

In some embodiments, the sample comprises 100 μM to 1M sodium triflinate. For example, in some embodiments the sample comprises 100 μM to 1M, 200 μM to 900 mM, 300 μM to 800 mM, 400 μM to 700 mM, 500 μM to 600 mM, 600 μM to 500 mM, 700 μM to 400 mM, 800 μM to 300 mM, 900 μM to 200 mM, or 1 mM to 100 mM sodium triflinate. In some embodiments, the sample comprises 1 mM to 100 mM sodium triflinate. For example, in some embodiments the sample comprises 1 mM to 100 mM, 5 mM to 95 mM, 10 mM to 90 mM, 15 mM to 85 mM, 20 mM to 80 mM, 25 mM to 75 mM, 30 mM to 70 mM, 35 mM to 65 mM, 40 mM to 60 mM, 45 mM to 55 mM, or about 50 mM sodium triflinate.

In some embodiments, the sample comprises the peroxide (e.g. TBHP, hydrogen peroxide) at a concentration of 100 μM to 1 M. For example, in some embodiments the sample comprises the peroxide at a concentration of 100 μM to 1 M, 200 μM to 900 mM, 300 μM to 800 mM, 400 μM to 700 mM, 500 μM to 600 mM, 600 μM to 500 mM, 700 μM to 400 mM, 800 μM to 300 mM, 900 μM to 200 mM, or 1 mM to 100 mM. In some embodiments, the sample comprises the peroxide at a concentration of 1 mM to 50 mM. For example, in some embodiments the sample comprises the peroxide at a concentration of 1 mM to −50 mM, 1 mM to 40 mM, 1 mM to 30 mM, 1 mM to 20 mM, or 1 mM to 10 mM.

In some embodiments, the sample additionally comprises a salt. In some embodiments, the sample comprises live cells containing the cell-membrane protein and a salt. In some embodiments, the salt comprises sodium chloride (NaCl), calcium chloride (CaCl₂)), potassium chloride (KCl), or sodium bicarbonate (NaHCO₃). In some embodiments, the sample comprises salt at a concentration such that the sample is isotonic with the live cells. In some embodiments, the sample comprises the salt at a concentration of 100 μM to 1 M. In some embodiments, the sample comprises the salt at a concentration of 5 mM to 250 mM. In some embodiments, the sample comprises the salt at a concentration of 25 mM to 150 mM. In some embodiments, the sample comprises the salt at a concentration of 140-160 mM. In some embodiments, the sample comprises the salt at a concentration of 145 mM.

In some embodiments, the method further comprises quenching the reaction after plasma generation. For example, in some embodiments the method further comprises quenching the sample with a solution comprising a radical quencher. In some embodiments, the radical quencher is a sulfur-containing amino acid, such as methionine, cystine, homocysteine, or taurine.

In some embodiments, the sample comprises one or more radical scavengers. Radical scavengers refer to any suitable substance that removes or deactivates free radicals in the sample. Suitable radical scavengers include, for example, methionine, cystine, homocysteine, taurine, DMSO, catalase, glucose, surfactants, buffers, histidine, DTT, TCEP, lipids, sucrose, etc. In some embodiments, the sample comprises DMSO.

The methods described herein can be utilized to determine structural information of a cell membrane protein(s) in a live, intact cell. Biological molecules can include secondary, tertiary, and quaternary structure that precludes solvent interaction with various parts of the biological molecule. In some embodiments, the methods described herein can be used to determine whether a portion of a cell membrane is accessible to a solvent. Labeling with a radical as described herein indicates that the portion of the biological molecule is accessible, whereas the absence of labeling indicates that the portion is inaccessible. In some embodiments, the methods can be used to assess a biological molecule having solvent accessible positions and solve inaccessible positions. In some embodiments, the methods can be used to identify binding epitopes on a cell membrane protein, such as epitopes that may interact with a drug or an agent when added to the cell. Cell membrane proteins represent targets for therapeutic agents/drugs, and as such information about binding epitopes obtained using the methods herein find use in methods of evaluating drug efficacy and in drug discovery.

The methods described herein can be useful for a variety of applications, including quality control for biopharmaceuticals, such as determining whether a biopharmaceutical has retained its secondary, tertiary, and/or quaternary structure. The methods described herein can be used in methods of assessing a disease state in a subject, such as diseases caused by a conformational change in one or more cell membrane proteins in a subject. For example, if a disease state is expressed by the breaking apart of a protein dimer, the methods of the present disclosure could be used to identify that the contact surfaces between the subunits of the dimer, which are normally not accessible to solvent, have become accessible to solvent. The methods described herein can also be utilized to study temperature-dependent properties of a sample of interest. For example, kinetics, protein folding, and other temperature-dependent mechanisms of interest can be studied with temperature-dependent deployment of the methods described herein. The methods described herein can be utilized to determine a rate of modification for components or sub-components within the sample of interest. For example, the methods described herein can compare the rate of oxidation (e.g. radical labeling) of two different residues on a protein of interest and can make various subsequent deductions based on the differences between those rates, such as determining a level of solvent accessibility.

### EXAMPLES

### Example 1

Membrane proteins account for a large portion of the proteome and are the targets for many drugs in development. However, mapping the HOS of membrane proteins using protein footprinting has presented a significant challenge due to the inherent insolubility and the presence of multiple transmembrane domains. Previous work has mostly focused on analyzing the soluble extracellular domain of membrane proteins. Recently, researchers have analyzed membrane proteins in vitro by embedding the full-length protein in an artificial membrane such as a vesicle or lipid nano disc, however, these systems do not fully account for the diversity of components and interactors present in a in vivo cell membrane. Another study utilized a different form of covalent labeling and diethyl pyrocarbonate (DEPC) for in vivo labeling, live-cell epitope mapping/footprinting of membrane bound tumor necrosis factor (mTNF) (Kirsch, Analytical Chemistry, 2023). While previous covalent labeling approaches such as DEPC expanded upon the modifiable residues available with standard HRF or OH labeling, a need clearly exists for platforms yielding a wider and robust coverage of labeling for in vivo protein footprinting.

Several existing challenges for effective in vivo protein footprinting include the low abundance and expression of membrane proteins. Herein, a platform for the expression and isolation of these proteins for downstream footprinting was developed. Notably, the in vivo labeling of these proteins is also exceedingly difficult. The lipid dense cell membrane represents a significant analytical challenge when it comes to radical based covalent labeling platforms, as it can scavenge radicals and decrease the amount of protein labeling. Additionally, buffer composition is to maintain pH during the oxidative labeling process, while also ensuring a proper ionic strength for cell viability, is needed. Varying sample conditions for the T-BRIMB-mediated radical footprinting platform can tune the chemistry and reactivity of the resulting labeling. Here, reaction conditions for the in vivo approach required special attention and care to further ensure maximum cell viability and labeling.

This example demonstrates for the first time successful use of plasma induced modification of molecules (PLIMB) to label proteins in vivo via hydroxyl radical footprinting. Specifically, the PLIMB-based hydroxyl radical footprinting platform was used in vivo with CTLA4-FLAG immunoprecipitated from FreeStyle™ 293-F cells and successful radical labeling of proteins was demonstrated.

### Materials and Reagents:

All chemicals and reagents were purchased from Sigma-Aldrich unless otherwise noted. Trypsin Platinum, Chymotrypsin, and 50× protease inhibitor cocktail were purchased from Promega. Optima LC-MS grade acetonitrile, water and 0.1% formic acid in water were purchased from Fisher Scientific.

### Experimental Cell Culture Conditions:

FreeStyle™ 293-F cells were cultured to mid-log phase (approximately 2.0×10⁶ cells/mL) in Gibco™ FreeStyle™ 293 Expression Medium. FreeStyle™ 293-F cells were transiently transfected with an expression plasmid containing an epitope-tagged antigen sequence with the TransIT-PRO® Transfection Reagent and incubated (37° C., 8% CO₂, 120 RPM, 48 hr). Cells were harvested via centrifugation (300×g, 3 min, RT) and washed with 50 mL PBS, pH 7.4 twice.

### Experimental Sample Preparation Condition:

HEK293 freestyle cells were resuspended 200 μL PBS, pH 7.4 and subjected to hydroxyl radical footprinting (HRF) via plasma induced modification of biomolecules (PLIMB). Specifically, antibody Mab (at a molar ratio of 1:1 Mab to antigen). Cells were subjected to 60s of submerged PLIMB (needle depth was 15 mm below surface). Cell viability was monitored to ensure cells were at least 90% viable before downstream protein isolation.

After the radical labeling, cells were centrifuged (300×g, 3 min, 4° C.) and the supernatant was discarded. Cells were solubilized by resuspending the cell pellet in 200 μL RIPA buffer [50 mM Tris-HCl, pH 7.8; 150 mM NaCl; 1% NP40 (v/v); 0.5% sodium deoxycholate (w/w); 0.1% SDS (w/w); 1× protease inhibitor cocktail, and 1 mM EDTA]. Cells were incubated (4° C., 1 hr, rotating end-over-end) to allow for full cell membrane solubilization and liberation of the epitope-tagged target antigen into the soluble fraction.

After the solubilization incubation, membranes and insoluble proteins were separated from the soluble fraction via centrifugation (15,000×g, 10 min, 4° C.). The 200 μL supernatant was added to 6.25 μL pre-equilibrated magnetic anti-epitope affinity resin and incubated (4° C., 1 hr, rotating end-over-end) to allow for immunoprecipitation of the epitope-tagged target antigen.

After the immunoprecipitation incubation, the magnetic beads were separated from the solution via a magnetic rack, and the supernatant was discarded. The magnetic beads were washed twice with 500 μL IP WASH I Buffer [10 mM sodium phosphate buffer, pH 7.5; 500 mM NaCl; 0.025% (v:v) Tween™-20; 1× protease inhibitor cocktail, and 1 mM EDTA]. The magnetic beads were washed four times with 500 μL IP WASH II Buffer [10 mM sodium phosphate buffer, pH 7.5; 500 mM NaCl]. For each wash, wash buffer was added to the magnetic beads, magnetic beads were incubated with the wash buffer (4° C., 2 min), magnetic beads were separated from the solution via a magnetic rack, and the supernatant was discarded.

Washed magnetic beads were then digested via addition of 50 μL 1.5 M GuHCl (in 50 mM Tris-HCl, pH 7.8; 5 mM DTT) and incubated (60° C., 10 min, 1000 rpm) to allow for the reduction and denaturation of the proteins bound to the beads. Denatured and reduced proteins were alkylated via addition of 1.5 μL of 500 mM IAA and incubated (RT, 30 min, in the dark). Alkylation reactions were quenched with the addition of 3 μL of 100 mM DTT. Proteins were digested via addition of 100 mM Tris-HCl, pH 7.8 with 1:10 trypsin platinum:protein (w:w) to a total volume of 200 μL and incubated overnight (37° C., 16 hr, 1000 rpm). Overnight trypsin digestions were further digested with 1:10 Chymotrypsin:protein (w:w) and incubated (37° C., 4 hr, 1000 rpm). Digested peptides were desalted via SPE and dried down in a SpeedVac.

### Experimental LC-MS/MS Conditions:

Dried-down peptides were resuspended into 0.1% formic acid injected into the LC/MS system for spectral analysis. The samples were analyzed using data-dependent acquisition with an Vanquish Neo coupled to an Orbitrap Elite 240 mass spectrometer (Thermo Scientific). A 30-minute chromatographic gradient from 2 to 40% acetonitrile with 0.1% formic acid was used for separation over a 2 μM, 15 cm Easy-Spray PepMap C18 column (ThermoFisher Scientific). A top-20 data-dependent acquisition was performed MS1 parameters of 120K resolving power in the Orbitrap, a scan range of 375-1200 m/z, a normalized AGC target of 300%, and MS² parameters of charge state 2-6 selection, a quadrupole isolation window of 2 Da, HCD collision energy of 30%, a normalized AGC value of 50%, and an automatic scan range starting at 110 m/z. Dynamic exclusion of 20 seconds was used after seeing an ion once. In cases where more coverage was needed a targeted or data-independent acquisition approach was utilized.

### Data Analysis for PLIMB Mediated OH Protein Footprinting:

The ‘.raw’ MS and MS/MS data files were searched against the protein fasta sequence using Protein Metrics Byos™ for peptide spectral matching and label-free quantification, with a 1% false discovery rate (FDR) cutoff. A list of standard expected modifications and radical mediated modifications were utilized in the database search. The following modifications were used in our search. Standard modifications: Carbamidomethyl/+57.021464 Da @ C (fixed). Variable modifications: Oxidation/+15.994915 Da @ C, F, H, M, W, Y, Dioxidation/+31.989829 Da @ C, F, M, W, Y, Cys-Oxidation/+15.994915−57.021464 Da @ C, Cys-Dioxidation/+31.989829−57.021464 Da @ C, Cys-Trioxidation/+47.984745−57.021464 Da @ C, Nitro/+44.985078 Da @ W, Y. The proportion of modified peptide (marked as expected variable modifications above) was calculated based on extracted ion chromatogram (XIC) relative peak areas of modified versus total peptide signal, including modified and unmodified peptides. Residue level quantification was utilized when chromatographic separation and sufficient flanking MS/MS fragments for the amino acid were present. All identifications and integrated areas were manually validated.

### Results

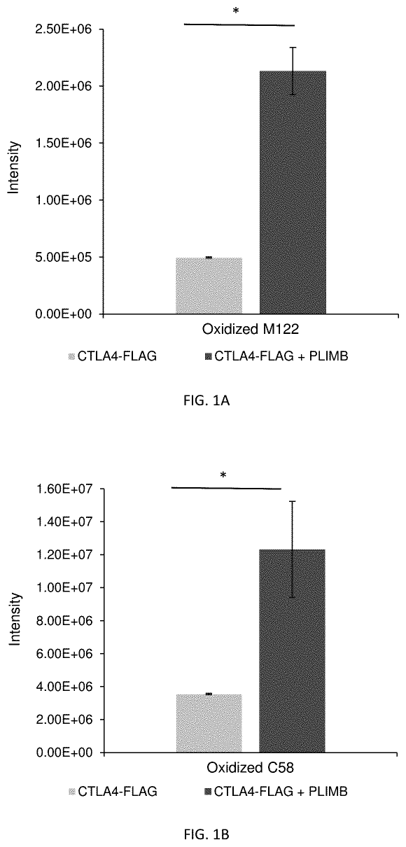

PLIMB-based hydroxyl radical footprinting was successfully used to label proteins in vivo with CTLA4-FLAG immunoprecipitated from FreeStyle™ 293-F cells. As shown in FIG. 1A, successful plasma induced labeling (e.g. oxidation) of methionine 122 from CTLA4-FLAG immunoprecipitated from FreeStyle™ 293-F cells occurred, and the level of oxidation is shown to be dependent on plasma exposure. As shown in FIG. 1B, successful plasma induced labeling (e.g. oxidation) of cysteine 58 from CTLA4-FLAG immunoprecipitated from FreeStyle™ 293-F cells occurred, and the level of oxidation is shown to be dependent on plasma exposure.

1. A method for in vivo labeling of cell membrane proteins with hydroxyl radicals, the method comprising:
a) providing a sample comprising one or more live cells; and
b) generating a plasma in the sample, thereby producing hydroxyl radicals in the sample which interact with cell membrane proteins associated with the one or more live cells, thereby labeling the cell membrane proteins in vivo.

2. The method of claim 1, wherein the sample comprises a phosphate buffer.

3. The method of claim 2, wherein the buffer comprises phosphate buffered saline (PBS) or sodium phosphate buffer.

4. The method of claim 3, wherein the sample comprises 1 mM to 500 mM PBS or sodium phosphate buffer.

5. The method of claim 4, wherein the sample comprises 1 mM to 50 mM PBS or sodium phosphate buffer.

6. The method of claim 5, wherein the sample comprises 5 mM PBS or sodium phosphate buffer.

7. The method of claim 1, wherein the sample additionally comprises a salt.

8. The method of claim 7, wherein the salt comprises sodium chloride (NaCl), calcium chloride (CaCl₂), potassium chloride (KCl), or sodium bicarbonate (NaHCO₃).

9. The method of claim 8, wherein the sample comprises salt at a concentration such that the sample is isotonic with the live cells.

10. The method of claim 7, wherein the sample comprises the salt at a concentration of 100 μM to 1M.

11. The method of claim 10, wherein the sample comprises the salt at a concentration of 5 mM to 250 mM.

12. The method of claim 11, wherein the sample comprises the salt at a concentration of 25 mM to 150 mM.

13. (canceled)

14. The method of claim 1, wherein the plasma is generated for 5 seconds to 120 seconds.

15. (canceled)

16. The method of claim 1, wherein plasma is generated from a plasma source point of a plasma electrode, wherein the plasma source point is (i) submerged within the sample, (ii) placed at an interface of a gas and the sample, (iii) placed above the sample at a distance of 10 cm or less from the sample, or (iv) placed above the sample at a distance of 3 cm or less from the sample.

17. The method of claim 16, wherein the plasma source point is submerged within the sample at a depth of 5-25 mm or at a depth of 15 mm.

18. (canceled)

19. (canceled)

20. The method of claim 16, wherein the gas is air.

21. (canceled)

22. (canceled)

23. The method of claim 1, further comprising quenching the sample with a solution comprising a radical quencher following generating the plasma in the sample.

24. The method of claim 1, wherein the sample further comprises a radical precursor for a second radical other than hydroxyl radicals, and wherein generating a plasma in the sample converts the radical precursor into the second radical which additionally interacts with and labels the cell membrane proteins in vivo.

25. The method of claim 24, wherein the second radical comprises trifluoromethyl radicals and wherein the radical precursor for the second radical comprises sodium triflinate.

26. (canceled)

27. The method of claim 1, further comprising identifying labeling of the biological molecule with the hydroxyl radicals using mass spectrometry.
